(19) United States
(12) Patent Application Publication
(10) Pub. No.: US 2011/0282192 A1
(43) Pub. Date: Nov. 17, 2011

(54) MULTIMODAL DEPTH-RESOLVING ENDOSCOPE

(76) Inventors: Noel Axelrod, Jerusalem (IL); Amir Lichtenstein, Tel-Aviv (IL); Gabby Sarusi, Rishon Le Zion (IL)

(21) Appl. No.: 13/146,955

(22) PCT Filed: Jan. 31, 2010
(86) PCT No.: PCT/IL10/00081

§ 371 Date: Jul. 29, 2011

(30) Foreign Application Priority Data
Jan. 29, 2009 (US) 61/148298

(51) Int. Cl. A61B 6/00

(52) U.S. Cl. 600/427

(57) ABSTRACT

A fiber-optic multimodal multi-spectral (MS), Optical Coherence Tomography (OCT), photoacoustic (PA) endoscope with beam scanning by a two-dimensional Microelectromechanical systems (MEMS) scanner present in the endoscopic head, combined in a synergetic way in a single endoscopic system. The PA, OCT and MS light sources are coupled to the endoscopic head through an optical switcher. Using a single optical endoscopic head and an electro-optical switch the endoscope of the invention is capable of sequential or parallel MS, OCT and PA imaging. The endoscope provides real-time imaging with a rate of 5 to 60 frames per second for each of the three imaging modalities.

### TECHNICAL FIELD

The present invention relates to endoscopy in general and to depth-resolving, multimodal endoscopy in particular.

### BACKGROUND ART

Endoscopy and Depth Resolved Endoscopy:

Endoscopy is a vital, yet minimally invasive, operative procedure, increasingly employed for both diagnosis and management of many medical and surgical conditions. There are broadly two classes of purely optical endoscope. The first endoscope developed in the 1960s uses a long flexible fiber-optic coupling between the remote lesion site and the user. This gives adequate diagnostic information, although the image quality may be compromised by both the number of (intact) elements within the fiber bundle and the light losses, which become significant when the individual fibers of a bundle decrease below 6-8 μm diameter.

An alternative class of endoscope uses a thin rigid tube enclosing a distributed lens system. These instruments are optically superior to their fiber bundle counterparts, although their use is confined to regions of the body that afford reasonably straight line access to the region of interest or keyhole surgical access, e.g. imaging ports in laparoscopic surgery.

The image contrast in these instruments originates from the surface and subsurface regions of the translucent tissue under examination.

For medical applications it is vital to discriminate between the structural features beneath the tissue surface. Depth-resolving imaging is required in surgery, neurosurgery, orthopedic, for dentist and oral surgeon, gynecology, cardiology, etc

Depth-resolving imaging is a well-established technique in biomedical imaging. This includes multi-spectral microscopy, two-photon microscopy, Optical Coherence Tomography (OCT), photoacoustic imaging and some other techniques. They differ in the physical principles of the underlying image contrast mechanism, image resolution and penetration depth.

Multi-spectral microscopy relies on optical contrast at different light wavelength, while OCT relies on optical scattering of ballistic photons and photoacoustic on optical absorption.

I. Single Fiber Multi-Spectral Microscope

One of the system modalities is an endoscopic multi-spectral microscopy that enables high resolution imaging of the surface at different wavelengths of light.

Multi-spectral imaging (MSI) is currently in a period of transition from its role as an exotic technique to its being offered in one form or another by all the major microscopy manufacturers. This is because it provides solutions to some of the major challenges in fluorescence-based imaging, namely ameliorating the consequences of the presence of autofluorescence and the need to easily accommodate relatively high levels of signal multiplexing. MSI, which spectrally characterizes and computationally eliminates autofluorescence, enhances the signal-to-background dramatically, revealing otherwise obscured targets. Some technologies used to generate multispectral images are compatible with only particular optical configurations, such as point-scanning laser confocal microscopy. Band-sequential approaches, such as those afforded by liquid-crystal tunable filters (LCTFs), can be conveniently coupled with a variety of imaging modalities, which, in addition to fluorescence microscopy, include brightfield (nonfluorescent) microscopy as well as small-animal, noninvasive in-vivo imaging. Brightfield microscopy is the chosen format for histopathology, which relies on immunohistochemistry to provide molecularly resolved clinical information. However, in contrast to fluorescent labels, multiple chromogens, if they spatially overlap, are much harder to separate and quantitate, unless MSI approaches are used. In-vivo imaging is a rapidly growing field with applications in basic biology, drug discovery, and clinical medicine.

The sensitivity of fluorescence-based in-vivo imaging, as with fluorescence microscopy, can be limited by the presence of significant autofluorescence, a limitation which can be overcome through the utilization of MSI.

II. OCT: Optical Coherence Tomography

OCT is a well established imaging technology that produces high resolution cross-sectional images of the internal microstructure of living tissue. The superb optical sectioning ability of OCT, which is achieved by exploiting the short temporal coherence of a broadband (white) light source, enables OCT scanners to image microscopic structures in tissue at depths beyond the reach of conventional bright-field and confocal microscopes. Probing depths exceeding 2 cm have been demonstrated in transparent tissues, including the eye and the frog embryo. In the skin and other highly scattering tissues, OCT can image small blood vessels and other structures as deep as 1-2 mm beneath the surface.

III. Photoacoustic Imaging

Photoacoustic imaging, however, does not rely on ballistic photons for excitation; and ultrasonic waves have 2-3 orders of magnitude weaker scattering than optical waves in biological tissues. Consequently, photoacoustic imaging provides high resolution at relatively large imaging depth. Therefore, photoacoustic imaging combines the advantages of optical absorption contrast with ultrasonic spatial resolution for deep imaging beyond the ballistic regime.

Photoacoustic microscopy (PAM) is a hybrid technique that detects absorbed photons ultrasonically through the photoacoustic effect. When a short-pulsed laser irradiates biological tissues, wideband ultrasonic waves (referred to as photoacoustic waves) are induced as a result of transient thermoelastic expansion. The magnitude of the photoacoustic waves is proportional to the local optical energy deposition and, hence, the waves divulge physiologically specific optical absorption contrasts. As optical energy deposition is related to the optical absorption coefficients of pigments, concentrations of multiple pigments can be quantified for functional imaging by varying the laser wavelength.

Because ultrasonic scattering is two to three orders of magnitude weaker than optical scattering in biological tissues, ultrasonic imaging can provide better spatial resolution than pure optical imaging when the imaging depth is beyond one optical transport mean-free-path (˜1 mm).

Typically, PAM uses neodymium-doped yttrium aluminum garnet (Nd:YAG) laser with about 10-ns laser pulses to generate photoacoustic waves. Laser light at a designated wavelength is delivered through an optical fiber to the photoacoustic (PA) microscope scanner. The energy of each laser pulse is detected by a photodiode for calibration.

MEMS Scanning Module

The system is a flexible fiber optic endoscope with optical Microelectromechanical systems (MEMS) Scanning micromirror head (MEMS Head). The instrument provides optical MEMS scanning real-time photoacoustic imaging

MEMS have enabled the miniaturization of scanning mirrors for placement at the distal end of fiber-based scanning microendoscopes. Other applications for beam-scanning micromirrors include image displays and optical switches.

Decoupled two-axis scanning has been demonstrated using vertical combdrives in polysilicon and Silicon-on-Insulator (SOI) (see S. Kwon, V. Milanovic, and L. P. Lee, “Vertical combdrive based 2-D gimbaled micromirrors with large static rotation by backside island isolation,” IEEE J Quantum Electron. 10, 498-504 (2004); D. Lee and O. Solgaard. “Two-axis gimbaled microscanner in double SOI layers actuated by self-aligned vertical electrostatic combdrive,” in Solid-State Sensor, Actuator and Microsystems Workshop, Hilton Head Island, (2004), pp. 352-355)). SOI technology is attractive for fabrication of MEMS mirrors due to features such as flat mirror surface and relatively simple fabrication.

Previous Works in MEMS Scanning Confocal Endoscopy

A miniature scanning confocal microscope using two single-axis micromirrors was proposed and demonstrated by Dickensheets and Kino (D. L. Dickensheets and G. S. Kino, “Silicon-micromachined scanning confocal optical microscope,” J MEMS 7, 38-47 (1998)). This compact module achieved high resolution confocal imaging at 20 frames/second over a 90 μm×90 μm field of view with a 1.1 mm working distance and 0.25 numerical aperture. More recent advances in scanning technology for confocal microscopy include three-dimensional dimensional scanning deformable micromirrors (Y. Shao and D. L. Dickensheets. “MEMS three-dimensional scan mirror,” in SPIE MOEMS Display and Imaging Systems II (SPIE, San Jose, Calif., 2004), pp. 175-183) and micromirrors for dual-axes confocal microscopy (H. Ra, Y. Taguchi, D. Lee, W. Piyawattanametha, and O. Solgaard. “Two-dimensional MEMS scanner for dualaxes confocal in vivo microscopy,” in Tech. Digest of IEEE International Conference on MEMS, (IEEE, Turkey, 2006), pp. 862-865) and tandem video-rate confocal microscope (Watson T. F., Neil M. A. A., Juskaitis R., Cook R. J. Wilson T., “Video-rate confocal endoscopy.”, Journal of Microscopy, Vol. 207, Pt 1 July 2002, pp. 37-42).

MEMS scanning confocal microscopes have also been fabricated using electrostatically actuated microlenses for focusing and scanning. Many technologies have been explored to miniaturize confocal microscopes. High deflection MEMS scanners can provide fast scanning and high resolution imaging, using appropriate lens systems, in a compact package.

Previous Works in MEMS Scanning OCT Endoscopy

In addition to confocal microscopy, MEMS mirrors have also been used for endoscopic OCT. Ex vivo OCT imaging of rat bladder has been accomplished using a single-axis MEMS mirror. More recent advances in scanning technology for OCT microscopy include 3D imaging with two axes scanning SOI MEMS micromirror.

### SUMMARY OF INVENTION

It is an object of the present invention to provide a multimodal endoscope integrating PA, OCT and MSI modalities.

It is another object of the present invention to provide a multimodal endoscope capable of using all modalities simultaneously and switching between modalities in real-time without removing the inserted endoscope from the body.

It a further object of the present invention to provide a multimodal endoscope with depth-resolving imaging capabilities.

It is yet another object of the present invention to provide a multimodal endoscope offering biological and biochemical information regarding processes in a studied tissue.

It is yet a further object of the present invention to provide a multimodal endoscope offering information regarding the conductivity of a studied tissue.

It is yet another object of the present invention to provide a multimodal endoscope for the detection of undersurface pathologies and structures such as blood vessels, urine vessels etc that lie under the tissue surface, during minimal invasive surgery procedures thus providing a safety margin for the surgeon.

It is yet a further object of the present invention to provide a multimodal endoscope for the targeted in depth and site restricted photobleaching, ablation or surgery without harming the surface and the tissue lying outside the plan of the focused radiation, of a living inhomogeneous tissue either sectioned or of an intact living body.

It is yet another object of the present invention to provide a multimodal endoscope capable of providing video rate depth-resolving imaging with resolution sufficient to resolve blood vessels or urea channels.

It is yet a further object of the present invention to provide a multimodal endoscope emitting high intensity, wavelength selectable light for surgical procedures and medical treatments with same setup for multimodal imaging to ensure minimal volume and weight.

The present invention relates to a modular endoscope unit which is insertable into all bodily cavities and is comprised of a flexible and guidable tube, leading a laser irradiation into a miniature head that injects electromagnetic radiation onto its target and collects the returning electromagnetic radiation and acoustic transients.

The endoscope is intended for real time biomedical imaging in vivo and in situ of cells, living tissues, of organs and bodily cavities for diagnostic purposes, morphological, physiological and biochemical investigation. Thus the system is an instrument that bridges form and functions and allows following the dynamics of the living cells tissues and organs of the living body.

Both imaging and surgery capabilities are generated by an ultrashort laser pulses that generate single photon or multiple photon (MP) excitation (irradiation) which is focused on the desired target by a focusing system designed for focusing divergent incident light beams on a common point on the sample face or inside the tissue.

Alternatively the system may provide in-depth imaging of a thick sectioned tissue which is kept alive and must be kept intact.

The imaging system enables in-depth resolving capabilities for the detection of undersurface pathologies and structures such as blood vessels, urine vessels etc that lie under the tissue surface, during minimal invasive surgery procedures thus providing a safety margin for the surgeon.

Alternatively the endoscope may be used for the targeted in-depth and site restricted photobleaching, ablation or surgery without harming the surface and the tissue lying outside the plan of the focused radiation, of a living inhomogeneous tissue either sectioned or of an intact living body.

Multiphoton excitation imaging relates to high harmonic generation of photons interacting nearly simultaneously (within 10-¹⁸ sec) with a nonlinear medium (no inversion symmetry). Deep tissue imaging is achieved with the longer (IR) wavelengths (700-1000 nm) that scatter considerably less than the equivalent single photons (350-500 nm) and allows penetration into inhomogeneous tissue while photodamage is restricted to the focal plane where the incident rays meet to enable the high harmonic event to happen.

The present invention thus relates to a fiber-optic multimodal endoscope, comprising:

(i) an optical coherent tomography (OCT) module comprising: an OCT light source, a fiber-optic Michelson interferometer, and an OCT detector;

(ii) a photoacoustic (PA) module comprising: a short pulsed PA light source, optical fibers, a PA detector, and an ultrasound transducer;

(iii) an optical switcher; and

(iv) an endoscopic head,

wherein said PA light source and said OCT light source are coupled to said endoscopic head through said optical switcher, and said endoscopic head controls the PA light source and the OCT light source, so that the endoscopic head injects electromagnetic radiation onto a target and then collects returning electromagnetic radiation and acoustic transients from the target.

In certain embodiments, the multimodal endoscope further comprises a multi-spectral imaging (MSI) module comprising: a broadband light source, collimated optics, and a color CCD/CMOS camera focal plan array, wherein said broadband light source is coupled to said endoscopic head through said optical switcher.

In certain embodiments, the head comprises a Micro-Opto-Electro-Mechanical Systems (MOEMS) scanning module.

In certain embodiments, the multimodal endoscope provides in-depth images for the detection of undersurface pathologies or structures.

In certain embodiments, the in-depth images are 3-dimensional images of 1, 2, 3, 4, 5, 6, 7, 8, 9 or 10 millimeters (mm) under tissue surface pathologies or structures.

In certain embodiments, the undersurface pathologies or structures comprise: large and small blood vessels, urine vessels, major nerves, bile ducts, or cartilage.

In certain embodiments, the multimodal endoscope further comprises a high power short pulsed laser light source that generates single photon or multiphoton (MP) excitation which are focused on a desired target by a focusing system designed for focusing divergent incident light beams on a common point on a sample face or inside a tissue.

In certain embodiments, the multiphoton excitation is a two-photon excitation which provides deep tissue imaging via longer Infra-Red (IR) wavelengths of 700 nm to 1000 nm that scatter considerably less than the equivalent single photons of 350 nm to 500 nm wavelengths, allowing deeper penetration into inhomogeneous tissue, and wherein photodamage is restricted to the focal plane where the incident beams meet when two photons meet almost simultaneously within 10-18 seconds.

In certain embodiments, the targeted in-depth and site restricted photobleaching, ablation, dissection or surgery without harming the surface and the tissue lying outside the plan of the focused radiation, of a living inhomogeneous tissue either sectioned or of an intact living body.

In certain embodiments, the focusing is done through the usage of two micro-sized resonating mirrors moved by two autonomous microelectromechanical systems (MEMS), both MEMS enabling focusing the two incident beams on a target, fixedly or in a rastering fashion.

In certain embodiments, the rastering is achieved by the rapid scanning of the focused laser beam in two dimensions, the X and the Y axis; the X-axis micro mirror achieving a 10 MHz-15 MHz frequency while the slow Y axis achieves a 15 Hz-50 Hz frequency, thus a high video rate of 60 Hz. may be accomplished.

In certain embodiments, the PA, OCT and MSI modalities may operate simultaneously or be switched from one imaging modality to another using the optical switch without removing the endoscope inserted into a body.

In certain embodiments, the OCT light source is a swept source.

In certain embodiments, the fiber-optic Michelson interferometer comprises a 2×2 beam-splitter, an A-Scan or M-scan mirros, fiber optics and a MOEMS spectrometer.

### BRIEF DESCRIPTION OF DRAWINGS

FIG. 1 shows a schematic illustration of a multimodal endoscoping system according to one embodiment of the invention, combining Photoacoustic (PA), Optical Coherence Tomography (OCT) and Multi-Spectral (MS) imaging modalities, wherein all modalities are connected to an endoscopic head via fiber optics and an optical switcher.

FIG. 2 depicts a scheme of the Microelectromechanical Systems (MEMS) based multimodal endoscope head comprising a MEMS scanner shown in FIG. 1.

FIG. 3 is a schematic illustration of a forward-looking endoscopic Micro-Opto-Electro-Mechanical Systems (MOEMS) head module equipped with a MEMS scanner. All parts are designed to be aligned by location in tight tolerance polyimide tubing.

FIG. 4 shows a block diagram of the architecture of the Multimodal Endoscopic System of the invention comprising a MOEMS scanning module equipped with a MEMS scanner unit with control electronics (ASIC) that is responsible for synchronization of lasers and MEMS scanner. The output signal from the detectors goes to an image frame grabber and fed into an imaging algorithm to display a combined depth-resolved image of the tissue interrogated by the laser sources.

FIG. 5 is a scheme of a Fourier domain optical coherence tomography (FD-OCT) microscopy in an endoscope. A swept source outputs a light that is directed both to an endoscopic reference arm and to an endoscopic detection (sample) arm connected to the Multimode Endoscopic Scanning Head (MESH) system.

FIG. 6 shows a general scheme of an endoscope based photoacoustic mode of the invention.

### MODES FOR CARRYING OUT THE INVENTION

In the following detailed description of various embodiments, reference is made to the accompanying drawings that form a part thereof, and in which are shown by way of illustration specific embodiments in which the invention may be practiced. It is understood that other embodiments may be utilized and structural changes may be made without departing from the scope of the present invention.

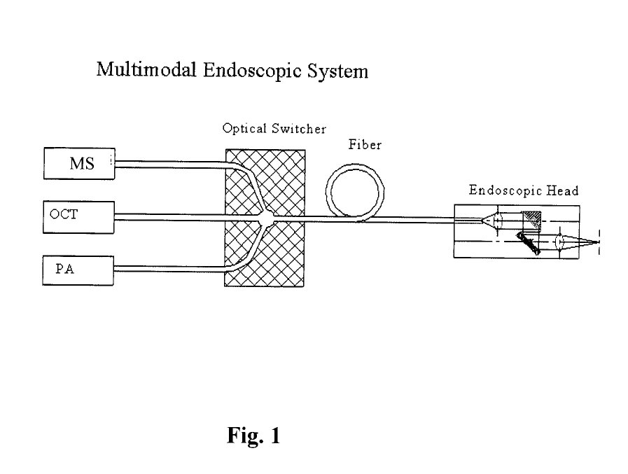

The present invention relates to a fiber-optic multimodal (multi-spectral, Optical Coherence Tomography, photoacoustic) endoscope with beam scanning by a two-dimensional (2D) MEMS scanner present in the endoscopic head. FIG. 1 shows the three imaging modalities (PA, OCT, and MS) combined in a synergetic way in a single endoscopic system. The PA, OCT and MS light sources are coupled to the endoscopic head through an optical switcher.

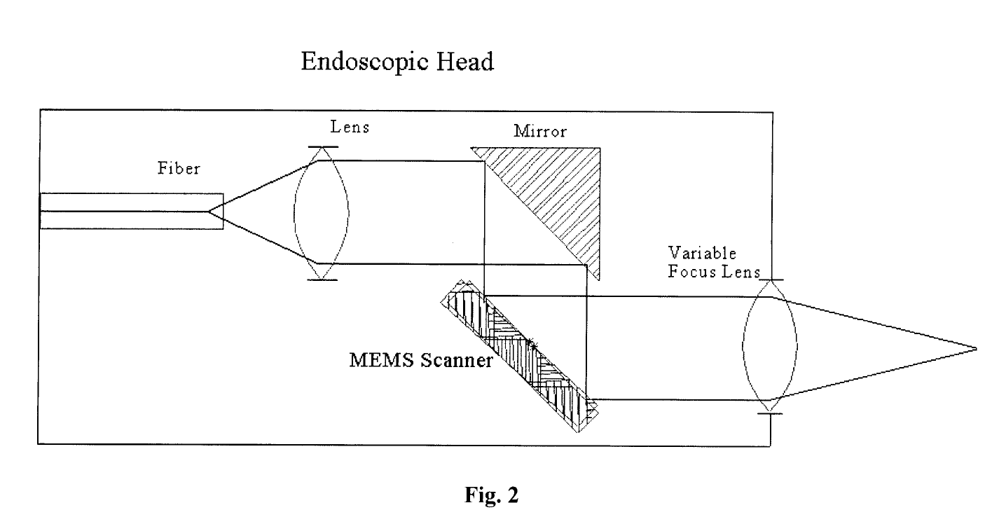

FIG. 2 is a close-up of the endoscopic head shown in FIG. 1. Using a single optical endoscopic head and an electro-optical switch the endoscope of the invention is capable of sequential or parallel multi-spectral, OCT and photoacoustic imaging. The endoscope provides real-time imaging with a rate of 5 to 60 frames per second for each of the three imaging modalities.

Multimode Endoscopic Scanning Head (MESH)

Optical System

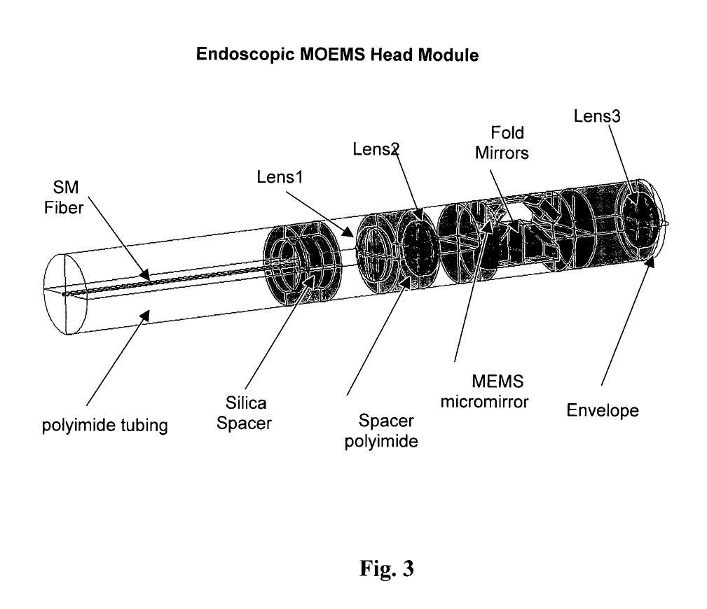

Reference is now made to FIG. 3 illustrating schematics of a forward-looking endoscopic head unit equipped with a MEMS scanner. The outer diameter of the tubing is about 4mm-6mm. All parts are designed to be aligned by location in tight tolerance polyimide tubing. The Silica Spacer is used for optical coupling between the single mode fiber and collimating lenses (shown as “Lens1” and “Lens2”). The Spacer polyimide is used as a mold material for the collimating lenses. The Fold Mirrors are used for redirecting the optical beam to a forward-looking configuration illustrated in FIG. 2 showing the light beam exiting from the endoscopic head in a straight line (and not sideways), on the side marked by “Envelope”.

The scanning head illustrated in FIG. 3 uses a single mode optical fiber (shown as “SM Fiber”) for the illumination.

The illumination light exiting the fiber is collimated by a pigtailed collimator to a beam diameter of about 0.5 mm, matched to the micromirror diameter. The MEMS micromirror scans the beam in the horizontal direction at the resonance frequency of the inner axis of the mirror, and in the vertical direction at a low frequency using the outer axis. An objective shown as “Lens3” (lens achromatic duplet) is used to form an image of the micromirror in the entrance aperture of the objective lens. These two lenses are selected to magnify the beam size and improve resolution without significantly compromising the scan angle and field of view.

An adaptive optics system can be obtained by placing a variable-focus lens at the exit aperture of the head. The optics of the MESH head is designed for minimization chromatic aberrations to allow broad band propagation. This is achieved by use of achromatic optics. Backscattered light is recollected and focused into the optical fiber by the same optical system.

MEMS Probe Package—Microscanner

Reference is now made to FIG. 2. The scanner used in the MESH is a suspended micromiRror with two-degree freedom. It is actuated by vertical, electrostatic combdrive actuators. The scanner is fabricated by performing deep-reactive ion etching (DRIE) process on a double SOI wafer. The mirror size may be about 500×500 μm². The inner axis of the mirror is controlled with an AC voltage signal at the resonance frequency of the mirror about 10-15 kHz, and the corresponding optical deflection is approximately ±6-9 degrees. The outer axis is actuated with a low frequency saw tooth wave at 10-50 Hz, with a voltage scan yielding an optical deflection of 6-9 degrees.

One of the key features of the proposed multimodal endoscope is use of a unit optical module (MESH system) for the three depth-resolving imaging modalities. The components (lasers, detectors, optics) for multi-spectral, OCT and photoacoustic modes are different but the endoscopic head used remains the same. The switching between different modes can be done in real time using optical switch technology between single mode fiber from a laser source of a given mode and the MESH system.

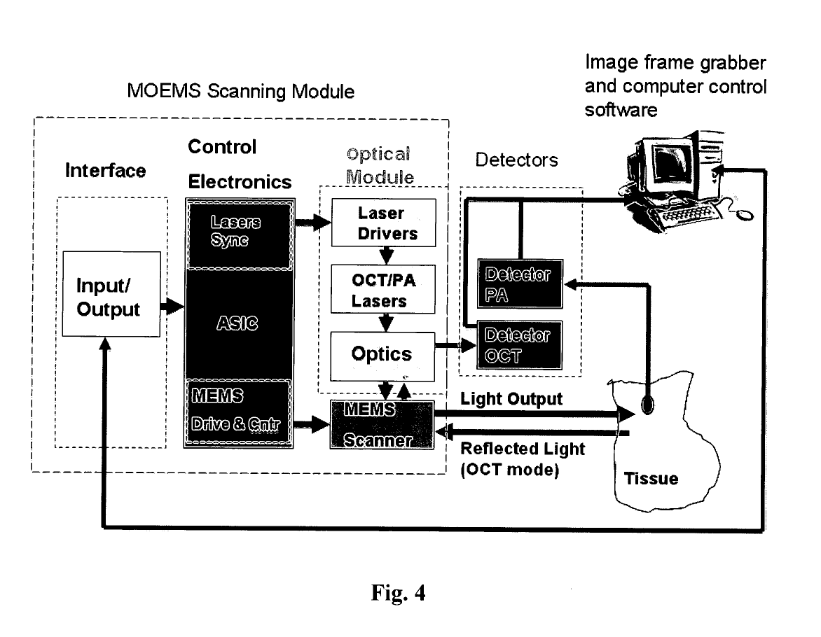

Reference is now made to FIG. 4 showing an embodiment of an architecture of the Multimodal Endoscopic System, which comprises a MOEMS scanning module with a MEMS scanner unit with control electronics (ASIC) that is responsible for synchronization of lasers (shown as “Lasers Sync” box) and driving and controlling (shown as the “MEMS Drive & Cntr” box) the MEMS scanner. The output signal from the detectors goes to an image frame grabber and an imaging algorithm to display a combined depth-resolved image of the tissue interrogated by the laser sources.

Input/Output is the driver and software interface between an external computer connected to the endoscope and the ASIC of the MOEMS scanning module. Switching between OCT/PA and multispectral imaging modalities is performed during procedure by the operator or automatically, via a controller in the external computer which in turn drives the optical switcher.

ASIC is the control electronics units (dye chip) which are a part of the MOEMS scanning module. The ASIC chip is electrically connected to the MEMS scanner, to the laser drivers and to the external computer. ASIC consists of the MEMS drive and control module that are responsible for synchronization of the scanning micromirror with the lasers and laser synchronization unit. The ASIC performs monitoring of the precise position of the mirror by use of MEMS position sensors. This allows achieving the desired accuracy in direction of the laser beams to obtain high resolution OCT/PA imaging. ASIC controls the OCT and PA laser sources (shown as “OCT/PA Lasers” box) through the laser drivers (shown as “Laser Drivers” box).

For imaging in the PA mode microsecond accuracy is needed to switch on/off laser beam output. At the same time the high accuracy is required for positioning micromirror about X and Y axis.

OCT/PA Optics (shown as “Optics” box in the Optical Module)—A laser beam from the PA/OCT source is coupled to the fiber by an optical collimator. The fiber is connected to the endoscopic head through the optical switcher. The endoscopic head (FIG. 2) comprises a single mode fiber, collimator lenses, static mirror, and objective micro lens.

The heater of the MEMS Scanner (shown as “MEMS Scanner” box) is the two axis scanning micromirror. The scan rate, amplitude and precise position are controlled by the ASIC.

The laser beam from the OCT source is focused on the tissue through the objective lens; the reflected light is collected by the same objective lens and is passed in the backward direction through the same optical path as in the forward direction and finally goes to the detector arm of the interferometer (2×2 coupler) to the photodetector (shown as “Detector OCT” box). The signal from the photodetector is collected by the data computer acquisition board and processed to form OCT image. The photodetector is a part of the endoscopic system but placed outside the endoscopic head.

The short-pulsed laser beam from PA source is focused through the collimator underneath the tissue surface. It generates a high-frequency ultrasound signal that is detected by the ultrasound detector (shown as “Detector PA” box). The ultrasound detector is a piezoelectric detector in the needle configuration; it is placed on the tissue side and electrically connected to the frame grabber of the computer (shown as “Image frame grabber and computer control software”).

Multispectral imaging: A light beam from a broadband source is passed through a tunable filter and collimated to the optical fiber. This light beam is coupled to the endoscopic head through the optical switcher. Inside the endoscopic head the light beam is projected through the same optical path as in the case of OCT mode. In contrast to the OCT mode the light beam is focused on the tissue surface. The focusing can be implemented by use of the variable focus lens objective. The light reflected from the tissue goes in a backward direction through the same optical path. It is collected by the photodetector. The detector is placed on the detector arm through a 2×1 beam-splitter outside the endoscopic head. The signal from the photodetector is acquired by the computer.

In addition to three imaging modalities, in two-photon absorption surgery a high-power very short-pulsed laser source is used. The light from the high-power very short-pulsed laser source is coupled through an optical collimator to the optical fiber. The light passes through the optical switcher to the endoscopic head. Inside the endoscopic head, the light passes through the same optical path as in case of other modalities and focuses on the interrogated tissue. The computer in this mode controls through the ASIC the exact direction of the light beam.

Detailed Description of Different Modalities

The section below provides a more detailed OCT, PA imaging modalities and two-photon absorption surgery description. The description of the multi-spectral imaging is omitted since this mode differs insignificantly from the visual mode implemented in existing endoscopes. The main difference from the visual mode is implementation of a tunable filter on the optical path length to acquire images at different wavelengths and image processing software that combines these images in the integrated way.

OCT mode—OCT is attracting interest among the medical community, because it provides tissue morphology imagery at much higher resolution (better than 10 μm) than other imaging modalities such as MRI or ultrasound.

The key benefits of OCT are:

1. Live sub-surface images at near-microscopic resolution;

2. Instant, direct imaging of tissue morphology;

3. No preparation of the sample or subject; and

4. No ionizing radiation.

Fourier domain OCT (FDOCT) measures interference fringes in the spectral domain to reconstruct an image tomography. Modulation of the interference fringe intensity in the spectral domain is used to determine the location of all scattering objects along the beam propagation direction. FDOCT offers significantly improved sensitivity and imaging speed compared to time domain OCT. Two methods have been developed to employ the Fourier domain technique: FDOCT using a spectrometer with a line-scan camera (G. Hausler and M. W. Linduer, J. Biomed. Opt. 3, 21 (1998)) and FDOCT using a rapidly swept laser source, i.e., “swept source OCT” (SSOCT) see (S. H. Yun, G. J. Tearney, J. F. de Boer, N. Iftimia, and B. E. Bouma, Opt. Express 11, 2953 (2003); or S. H. Yun, G. J. Tearney, J. F. de Boer, N. Iftimia, and B. E. Bouma, Opt. Express 11, 2953 (2003)). SSOCT has the advantage of a simple system setup, low cost, and capability of balanced detection. In addition, mirror image and autocorrelation noise can be removed instantaneously by the simple addition of an electro-optic modulator.

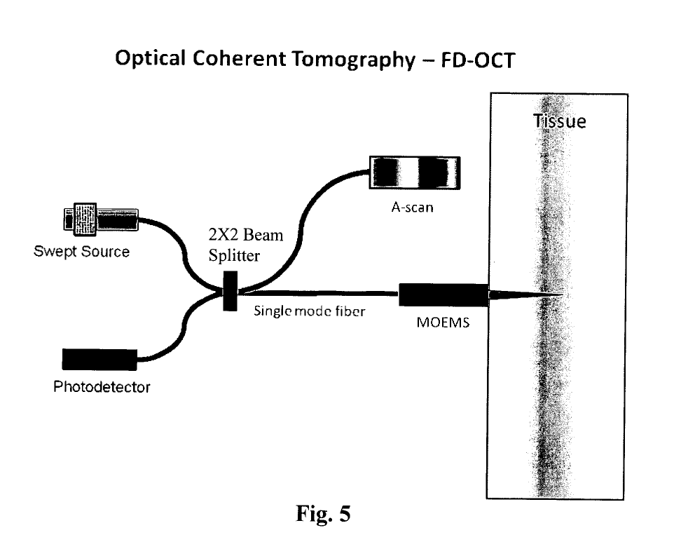

Reference is now made to FIG. 5 showing a schematic diagram of the FDOCT system based on the built swept source. The output light from the swept source is split into a reference arm and a detection (sample) arm by a 2×2 beam-splitter (also called coupler). The fiber optics, 2×2 beam splitter, A-Scan (amplitude modulation scan) and MOEMS are referenced together as a Michelson interferometer.

The reference arm comprising an A-Scan unit is used to scan optical pathlength in the reference arm of the Michelson interferometer, rapidly and precisely. The pathlength must be varied over a distance large enough to cover the desired axial imaging range, which may be as large as a centimeter or more for ocular imaging and 2 mm for imaging skin and other optically dense tissues, and its positioning inaccuracy must be a fraction of the source coherence length. The desired scanning speeds can be attained by using a piezoelectric transducer to drive a parallel minor system in which light reflects multiple times (Y. Pan, E. Lankenau, J. Welzel, R. Birngruber, and R. Engelhardt, “Optical coherence-gated imaging of biological tissues,” IEEE J. Select. Topics Quantum Electron., vol. 2, pp. 1029-1034, 1996).

In the detection arm, the signal from the balanced detector is converted by a data acquisition board. The number of data points for each A-line data acquisition during the frequency scan is about 1,000. The detected fringe signal is transformed from time to frequency domain with the swept spectral function (R. Huber, M. Wojtkowski, K. Taira, J. G. Fujimoto, and K. Hsu, Opt. Express 13, 3513 (2005)). The OCT image is formed from the processed spectral signal at each pixel of the focus plane.

Photoacoustic Mode

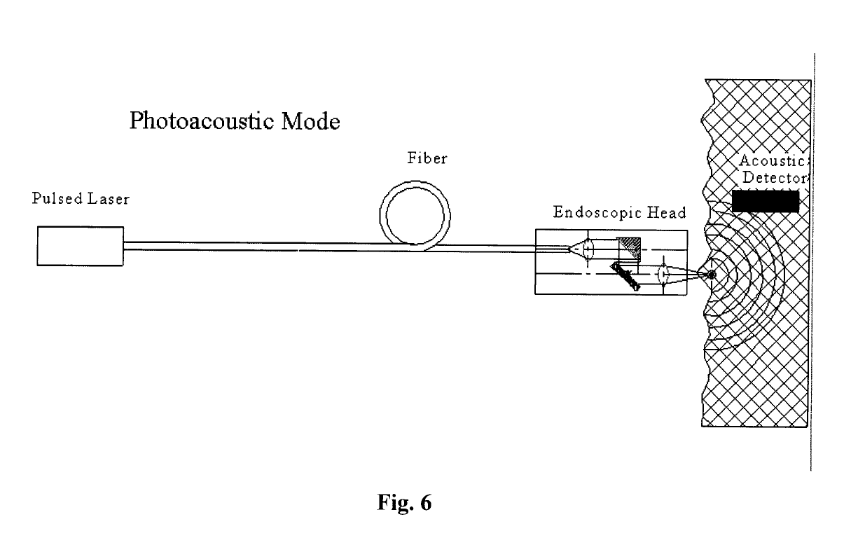

Reference is now made to FIG. 6 showing a general scheme of an endoscope based photoacoustic mode (PAM) of the invention. In PAM systems, 10-ns laser pulses (shown as “Pulsed Laser” box) from a tunable dye laser pumped by a Nd:YAG laser (or mode-locked fiber-laser in other arrangement) are used to generate photoacoustic waves. Laser light at a designated wavelength is delivered through an optical fiber (shown as “Fiber”) to the endoscopic MESH system (shown as “Endoscopic Head” and shown in more detail in FIG. 3). The energy of each laser pulse is detected by a photodiode for calibration. The laser beam from the fiber is weakly focused into the tissue to avoid tissue overheating. It is achieved by employing adaptive optics of the MESH system. The generated photoacoustic wave is collected by ultrasonic transducer (or transducers) that is in contact with the tissue about 2-10 cm away from the irradiating region. The transducers can be a part of the endoscopic head (to be placed at the tip of the endoscopic head and thus in contact with the tissue). In other arrangements, a needle-type ultrasonic hydrophone with the bandwidth up to 20 MHz is inserted into the tissue. In an optically clear medium, the optical focus of the laser beam is about 100 μm in diameter. In certain embodiments, the ultrasonic transducer or transducers are used with a large numerical aperture (NA) ultrasound lens, a high central frequency and a wide bandwidth for achieving high spatial resolution. However, at central frequencies higher than 10 MHz, it is the penetration depth of ultrasound, rather than the penetration depth of light, that limits the maximum imaging depth owing to strong frequency-dependent acoustic attenuation.

The system provides a raster scanning of lateral (x-y) plane with a step size of about 100 μm. The scan area is about 2×2 mm (3×3 mm for 9⁰ tilt). The pulse repetition rate is about 325 kHz to achieve 8 Hz frame rate.

The high frequency scanner mirror can be used to study axon activity and metabolism as a tool in learning neuron physiology and pathology, thus providing information about tissue conductivity.

The high frequency scanner mirror can be used to study axon activity and metabolism as a tool in learning neuron physiology and pathology, thus providing information about tissue conductivity.

Two Photon Absorption Surgery and Imaging Method

Another technique that can be used with the endoscope according to the invention is the two-photon absorption surgery and imaging method. An object of this technique is to acquire tissue ablation for microsurgery. This can be achieved by the two-photon excitation phenomenon whereby a chemical group capable of selectively absorbing a specific lightwave (chromophore) absorbs two photons nearly simultaneously (within 10⁻¹⁸ sec), where each photon is twice the wavelength (half the energy) of a single photon needed to excite the chromophore. This process is termed nonlinear excitation since when a fluorophore is excited it emits with intensity which is proportional to the square of the excitation intensity (three photons emission is cubed). This nonlinear relationship is used to generate an in depth sectioning because it is generated at the focal point and it falls off rapidly away from this point. The light source is a high-power, short pulsed laser such as the Ti-sapphire laser.

The probability of the simultaneous absorption is proportional to the product of the pulse repetition rate and the pulse duration. Thus shortening the pulse duration and/or reducing the pulse rate increases the probability of two photons generation. For example, in mode locked Ti-sapphire laser pulse duration is ˜100 fsec.

The advantages are distinct: the longer infrared (IR) photons (700-1000 nm) are less damaging and induce less phototoxic effects in the cells. They scatter considerably less than the shorter ones (350-500 nm) allowing deeper penetration into inhomogeneous tissue. This enables optical sectioning and deep penetration within thick tissue by targeting the volume of the tissue to be ablated at a required depth by the focusing of two lower energy photons that together produce a very damaging ultraviolet (UV) wavelength (<300 nm).

Directing of the ablation beam is done by the same MEMS micromirror that is also used for imaging (confocal, photoacoustic, OCT). Focusing is done by the variable focus length lens. The advantage of the two-photon absorption method for surgery is that the two-photon event occurs only in illuminating a small volume at the focal point instead of the hourglass volume usually achieved by a single photon, thus avoiding one of the drawbacks of confocal microscopy, i.e., the excitement of the specimen above and below the focal plane. In two-photon microscopy, a volume lesser than 1 femtoliter with <1-μm resolution in the Z direction was achieved with a theoretically sub-cellular resolution.

The advantages of this method can be deduced from the advantages two photon imaging has over single photon excitation. The longer infrared wavelength scatter considerably less than the shorter wavelengths allowing deeper penetration with lesser photoxicity. In the intact brain, imaging depth of 0.5-1 mm were achieved bringing most layers of cortex as well as underlying circulatory system and superficial structures within reach. Gradient index lenses (GRIN) might be used for this purpose.

Another innovative application is imaging of the retina, in which the two-photon focusing technique is able to excite a chromophore present only at the focal point, whereas same photoreceptors do not respond to longer IR wavelengths above or below ablation of damaged tissue at the bottom of the retina might be produced. Another application of two-photon microscopy is two-photon photolysis of trapped species that, when excited by light, turn from being inert to active. For example, immediate release of Ca⁺² ions from the photolabile calcium chelator DM-nitrophen (Parthasarathy. K, (2006), J. Clin. Invest., 116: 2193-2200). Rapid release of calcium ions from its cages was exploited to investigate the role of intracellular Ca⁺² microdomains in regulation of calcium ions sensitive processes.

The multimodal endoscope of the present invention has many applications in diagnostics and surgery in different field of Medicine. For example, in cancer surgery, where it can show the tumor borders and demonstrate neovascularization and navigate the surgeon such as to avoid damaging vital structures; in orthopedics, to show cartilage smoothness, early damage to connective tissue, or ligaments with internal (undersurface) damages; to show to the dentist or oral surgeon the abscess and his fistula online; to measure the sinus and the bone thickness; to demonstrate nerve bundle for preventing damage. To demonstrate middle ear infection and differentiate inner ear tiny structure; in Cardiology, to identify plaque in the blood vessel and its content, to demonstrate the depth of damage during ablation and to help visualization of small vessels; in surgery in general, to prevent damaging of vital organs and better demonstrate the operative plan, for example, by helping to identify the clean edge of tumor borders; in Gynecology, to prevent damage to the urether and other vital organs and to offer the surgeon better planning and safety of the operation; in Urology, to improve the view of the bladder, to improve the resection of the prostate and show more safer way to prevent from harming nerves during radical procedure; in plastic surgery, the endoscope technique of the invention can lead to better cosmetic result, due to its resolution capabilities.

The multimodal endoscope of the invention can be used for detecting one or more of the following pathologies:
(i) hallmarks of cancers, including angiogenesis, hyper-metabolism and pleomorphism, enabling the detection and study of early cancers;
(ii) utilizing the spectroscopic differences between oxy- and deoxy-hemoglobin, one can infer on the distribution of oxygen in the intact tissue and organ, enabling the assessment of physiological and functional properties of the brain, to assess the effectiveness of chemotherapy on cancerous tissue, to measure differences in vascularization, tissue diffusion properties and site specific oxygen consumption, to understand and assess the integrity and viability and function of an organ;
(iii) to assess architectural changes at the cellular and sub-cellular levels via analysis of the optical scattering properties of; and
(iv) functional information of living tissues at the molecular or genetic level can be revealed by optical imaging by employing exogenous bioactive optical contrast agents.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments.

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a sub-combination or variation of a sub-combination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what essentially incorporates the essential idea of the invention.

1. A fiber-optic multimodal endoscope, comprising:
(i) an optical coherent tomography (OCT) module comprising: an OCT light source, a fiber-optic Michelson interferometer, and an OCT detector;
(ii) a photoacoustic (PA) module comprising: a short pulsed PA light source, optical fibers, a PA detector, and an ultrasound transducer;
(iii) an optical switcher; and
(iv) an endoscopic head,
wherein said PA light source and said OCT light source are coupled to said endoscopic head through said optical switcher, and said endoscopic head controls the PA light source and the OCT light source, so that the endoscopic head injects electromagnetic radiation onto a target and then collects returning electromagnetic radiation and acoustic transients from the target.

2. A multimodal endoscope according to claim 1, further comprising a multi-spectral imaging (MSI) module comprising: a broadband light source, collimated optics, and a color CCD/CMOS camera focal plan array, wherein said broadband light source is coupled to said endoscopic head through said optical switcher.

3. A multimodal endoscope according to claim 1, wherein said endoscopic head comprises a Micro-Opto-Electro-Mechanical Systems (MOEMS) scanning module.

4. A multimodal endoscope according to claim 1, providing in-depth images for the detection of undersurface pathologies or structures.

5. A multimodal endoscope according to claim 4, wherein said in-depth images are 3-dimensional images of 1, 2, 3, 4, 5, 6, 7, 8, 9 or 10 millimeters (mm) under tissue surface pathologies or structures.

6. A multimodal endoscope according to claim 4, wherein said undersurface pathologies or structures comprise: large and small blood vessels, urine vessels, major nerves, bile ducts, or cartilage.

7. A multimodal endoscope according to claim 1, further comprising a high power short pulsed laser light source that generates single photon or multiphoton (MP) excitation which are focused on a desired target by a focusing system designed for focusing divergent incident light beams on a common point on a sample face or inside a tissue.

8. A multimodal endoscope according to claim 7, wherein said multiphoton excitation is a two-photon excitation which provides deep tissue imaging via longer Infra-Red (IR) wavelengths of 700 nm to 1000 nm that scatter considerably less than the equivalent single photons of 350 nm to 500 nm wavelengths, allowing deeper penetration into inhomogeneous tissue, and wherein photodamage is restricted to the focal plane where the incident beams meet when two photons meet almost simultaneously within 10-¹⁸ seconds.

9. A multimodal endoscope according to claim 7, for targeted in-depth and site restricted photobleaching, ablation, dissection or surgery without harming the surface and the tissue lying outside the plan of the focused radiation, of a living inhomogeneous tissue either sectioned or of an intact living body.

10. A multimodal endoscope according to claim 7, wherein said focusing is done through the usage of two micro-sized resonating mirrors moved by two autonomous microelectromechanical systems (MEMS), both MEMS enabling focusing the two incident beams on a target, fixedly or in a rastering fashion.

11. A multimodal endoscope according to claim 10, wherein said rastering is achieved by the rapid scanning of the focused laser beam in two dimensions, the X and the Y axis; the X-axis micro mirror achieving a 10 MHz-15 MHz frequency while the slow Y axis achieves a 15 Hz-50 Hz frequency, thus a high video rate of 60 Hz. may be accomplished.

12. A multimodal endoscope according to claim 2, wherein the PA, OCT and MSI modalities may operate simultaneously or be switched from one imaging modality to another using the optical switch without removing the endoscope inserted into a body.

13. A multimodal endoscope according to claim 1, wherein said OCT light source is a swept source.

14. A multimodal endoscope according to claim 1, wherein said fiber-optic Michelson interferometer comprises a 2×2 beam-splitter, an A-Scan or M-scan mirros, fiber optics and a MOEMS spectrometer.
